(19) United States
(12) Patent Application Publication
(10) Pub. No.: US 2018/0008518 A1
(43) Pub. Date: Jan. 11, 2018

(54) MINERALIZATION FLUORIDE COMPOSITIONS

(71) Applicant: THE UNIVERSITY OF MELBOURNE, Victoria (AU)

(72) Inventor: Eric Charles Reynolds, Carlton, Victoria (AU)

(73) Assignee: THE UNIVERSITY OF MELBOURNE, Victoria (AU)

(21) Appl. No.: 15/538,836

(22) PCT Filed: Dec. 24, 2015
(86) PCT No.: PCT/AU2015/050843

§ 371 Date: Jun. 22, 2017

(30) Foreign Application Priority Data
Dec. 24, 2014 (AU) 2014905281

(51) Int. Cl. A61K 8/24; A61K 6/033; A61K 6/00; A61K 8/02; A61Q 11/00; A61K 8/21

(52) U.S. Cl. CPC A61K 8/24; A61Q 11/00; A61K 8/21; A61K 6/0017; A61K 8/0216; A61K 6/033; A61K 2800/92

(57) ABSTRACT

The present invention relates to compositions for mineralizing a dental surface, in particular tooth enamel. Methods of mineralizing hypomineralized lesions (including subsurface lesions) in the tooth enamel caused by dental caries, dental corrosion and fluorosis are also provided.

### FIELD OF THE INVENTION

The present invention relates to compositions for uses including mineralizing a dental surface, in particular tooth enamel. Methods of mineralizing hypomineralized lesions (including subsurface lesions) in the tooth enamel caused by various means including dental caries, dental corrosion and fluorosis are also provided.

### CROSS REFERENCE(S) TO RELATED APPLICATIONS

This application claims priority from Australian provisional patent application no. 2014905281, the entire disclosure of which is incorporated herein by reference.

### BACKGROUND OF THE INVENTION

Common causes of hypomineralized lesions are caries and fluorosis.

Dental caries result from the demineralization of hard tissue of the teeth usually because of fermentation of dietary sugar by dental plaque odontopathogenic bacteria. Dental caries is still a major public health problem. Further, restored tooth surfaces can be susceptible to further dental caries around the margins of the restoration. Even though the prevalence of dental caries has decreased through the use of fluoride in most developed countries, the disease remains a major public health problem. Dental erosion or corrosion is the loss of tooth mineral by dietary or regurgitated acids. Dental hypersensitivity is due to exposed dentinal tubules through loss of the protective mineralized layer, cementum. Dental calculus is the unwanted accretion of calcium phosphate minerals on the tooth surface. All these conditions, dental caries, dental erosion, dental hypersensitivity and dental calculus are therefore related to imbalances in the level of calcium phosphates.

Enamel fluorosis (mottling) has been recognized for nearly a century, however, the aetiological role of fluoride was not identified until 1942. The characteristic appearance of fluorosis may be differentiated from other enamel disturbances. The clinical features of fluorotic lesions of enamel (FLE) represent a continuum ranging from fine opaque lines following the perikymata, to chalky, white enamel. The presence of a comparatively highly mineralized enamel outer surface and a hypomineralized subsurface in the fluorotic lesion stimulates the incipient enamel “white spot” carious lesion. With increasing severity, both the depth of enamel involved in the lesion and the degree of hypomineralization increases. The development of fluorosis is highly dependent on the dose, duration and timing of fluoride exposure and is believed to be related to elevated serum fluoride concentrations. Chalky “white spot” lesions may also form on developing teeth in children such as after treatment with antibiotics or fever. Such lesions indicate areas of hypomineralization (i.e. too little mineralization) of the tooth enamel.

Depending on lesion severity, fluorosis has been managed clinically by restorative replacement or micro-abrasion of the outer enamel. These treatments are unsatisfactory because they involve restorations or removal of tooth tissue. What is desired is a treatment that will mineralize the hypomineralized enamel to produce a natural appearance and structure.

Specific complexes of casein phosphopeptides and amorphous calcium phosphate (“CPP-ACP”, available commercially as Recaldent™) have been shown to remineralize enamel subsurface lesions in vitro and in situ (Reynolds, 1998; Shen et al., 2001; Reynolds et al., 2003).

WO 98/40406 in the name of The University of Melbourne (the contents of which are herein incorporated fully by reference) describes casein phosphopeptide-amorphous calcium phosphate complexes (CPP-ACP) and CPP-stabilized amorphous calcium fluoride phosphate complexes (CPP-ACFP) which have been produced at alkaline pH. Such complexes have been shown to prevent enamel demineralization and promote remineralization of enamel subsurface lesions in animal and human in situ caries models (Reynolds, 1998). Improved casein phosphopeptide-amorphous calcium phosphate complexes (CPP-ACP) and CPP-stabilized amorphous calcium fluoride phosphate complexes (CPP-ACFP) have also been described in WO2006/056013 and WO2006/135982, including preferred complexes formed at a pH of 5 to 6.5.

The CPP which are active in forming the complexes do so whether or not they are part of a full-length casein protein. Examples of active (CPP) that can be isolated after tryptic digestion of full length casein have been specified in U.S. Pat. No. 5,015,628 and include peptides Bos αₛ₁-casein X-5P (f59-79), Bos β-casein X-4P (f1-25), Bos αₛ₂-casein X-4P (f46-70) and Bos αₛ₂-casein X-4P (f1-21).

WO 2010/068359 describes treating calcium phosphate particles with at least one sugar alcohol and/or at least one glycerophosphoric acid compound.

There is a need to provide improved or alternative treatments for hypomineralized lesions.

Reference to any prior art in the specification is not, and should not be taken as, an acknowledgment or any form of suggestion that this prior art forms part of the common general knowledge in Australia or any other jurisdiction or that this prior art could reasonably be expected to be ascertained, understood and regarded as relevant by a person skilled in the art.

### SUMMARY OF THE INVENTION

In one aspect, the present invention provides a method of mineralizing a dental surface or sub-surface comprising contacting the dental surface or subsurface with calcium monofluorophosphate and a mineralizing agent.

The calcium monofluorophosphate (also referred to herein as CaMFP or CaFPO₃⁰) may be present in, or provided in, an amount outlined below. For example, calcium monofluorophosphate may be present in a composition of the invention or used in a method of the invention at about 200 ppm to 5000 ppm F. As explained further below, it is common to specify fluoride content by ppm—as each CaMFP has one fluoride, the same measurement is appropriate. In a preferred embodiment, calcium monofluorophosphate is present in an amount of about 200 ppm to about 3000 ppm F, 400 ppm to about 1500 ppm F, or about 1000 ppm to 1450 ppm F. In a further preferred embodiment, calcium monofluorophosphate is present in an amount of about above 500, 900, 1000, 1100, 1200, 1300, 1400 or 1500 ppm F.

Preferably, the mineralizing agent is stabilized amorphous calcium phosphate (ACP) and/or amorphous calcium fluoride phosphate (ACFP). The dental surface is preferably dental enamel. In one embodiment the dental sub-surface is a lesion in the enamel, such as a lesion caused by caries, dental erosion or fluorosis.

ACP and ACFP can precipitate at therapeutic concentrations, so preferably the ACP and/or ACFP is phosphopeptide (PP)-stabilized. Preferably, the phosphopeptide (as defined below) is a casein phosphopeptide. Preferably, the ACP or ACFP is in the form of a casein phosphopeptide stabilized ACP or ACFP complex.

In a preferred embodiment, the phosphopeptide stabilized amorphous calcium phosphate (ACP) or amorphous calcium fluoride phosphate (ACFP) complex has tightly bound and loosely bound calcium, wherein the loosely bound calcium in the complex is less than the tightly bound calcium in an ACP or ACFP complex formed at a pH of 7.0. Optionally, the ACP or ACFP is predominantly in a basic form.

In a preferred embodiment, the calcium ion content of the stabilized ACP or ACFP complex is in the range of about 30 to 100 moles of calcium per mole of PP. More preferably, the calcium ion content is in the range of about 30 to about 50 moles of calcium per mole of PP.

In any aspect or embodiments as described herein, the stabilized ACP and/or ACFP may be in a formulation with additional calcium salt (e.g. lactate, carbonate, phosphate, chloride etc). Typically, the formulation includes a PP stabilized ACP and/or ACFP complex together with at least an equal amount by weight of soluble calcium phosphate.

Preferably, the phase of the ACP is predominantly a basic phase, wherein the ACP comprises predominantly the species Ca²⁺, PO₄³⁻ and OFF. The basic phase of ACP may have the general formula [Ca₃(PO₄)₂]ₓ[Ca₂(PO₄)(OH)] where x≧1. Preferably x=1-5. More preferably, x=1. Preferably the two components of the formula are present in equal proportions. Accordingly, in one embodiment, the basic phase of ACP has the formula Ca₃(PO₄)₂Ca₂(PO₄)(OH).

Preferably, the phase of the ACFP is predominantly a basic phase, wherein the ACFP comprises predominantly the species Ca²⁺, PO₄³⁻ and F. The basic phase of ACFP may have the general formula [Ca₃(PO₄)₂]ₓ[Ca₂(PO₄)F]_{y} where x≧1 when y=1 or where y≧1 when x=1. Preferably, y=1 and x=1-3. More preferably, y=1 and x=1. Preferably the two components of the formula are present in equal proportions. Accordingly, in one embodiment, the basic phase of ACFP has the formula Ca₃(PO₄)₂Ca₂(PO₄)F.

In one embodiment, the ACP complex consists essentially of phosphopeptides, calcium, phosphate and hydroxide ions and water.

In one embodiment, the ACFP complex consists essentially of phosphopeptides, calcium, phosphate, fluoride and hydroxide ions and water.

In a further aspect of the present invention there is provided a method of mineralizing a dental surface comprising providing calcium monofluorophosphate and a source of ACP or ACFP. In a preferred embodiment the dental surface is enamel.

In a further aspect of the present invention there is provided a method of treating fluorosis comprising contacting a fluorotic lesion in tooth enamel with calcium monofluorophosphate and stabilized ACP and/or ACFP.

In a further aspect of the present invention there is provided a method of treating dental caries comprising contacting a caries lesion in tooth enamel with calcium monofluorophosphate and stabilized ACP and/or ACFP.

In a further aspect of the present invention there is provided a method of treating dental erosion comprising contacting a lesion in tooth enamel caused by erosion with calcium monofluorophosphate and stabilized ACP and/or ACFP.

In a further aspect of the present invention there is provided a method of reducing white spot lesions on the tooth enamel comprising contacting a white spot lesion with calcium monofluorophosphate and stabilized ACP and/or ACFP.

In a further aspect of the present invention there is provided a method of remineralizing a lesion in tooth enamel comprising contacting the lesion with calcium monofluorophosphate and stabilized ACP and/or ACFP.

In a further aspect of the invention there is provided a method for remineralizing a lesion in tooth enamel comprising:

contacting the lesion with calcium monofluorophosphate and a source of ACP or ACFP; and

administering a composition which is capable of increasing or maintaining the pH of a solution.

Preferably the composition which is capable of increasing or maintaining the pH of a solution contains sodium bicarbonate or urea. Preferably, the composition is a mouthrinse or mouthwash.

In one embodiment, the compound which is capable of increasing or maintaining the pH of a solution is not NaOCl, i.e. the composition of the invention is NaOCl free.

In any aspect or embodiment of the invention described herein, the compound which is capable of increasing or maintaining the pH of a solution is administered concurrently with, as a pre-treatment to, or as a post-treatment to a source of stabilized ACP or ACFP and calcium monofluorophosphate.

In any aspect or embodiment of the invention described herein, the ACP and/or ACFP is phosphopeptide (PP)-stabilized. Preferably, the phosphopeptide (as defined below) is a casein phosphopeptide. Preferably, the ACP or ACFP is in the form of a casein phosphopeptide stabilized ACP or ACFP complex.

In another aspect there is provided a composition including stabilized amorphous calcium phosphate (ACP) and/or amorphous calcium fluoride phosphate (ACFP) and calcium monofluorophosphate. The composition preferably further comprises a compound increasing or maintaining the pH of a solution as described herein.

The stabilized ACP and/or ACFP and/or calcium monofluorophosphate may be contacted with the dental surface for a period of about 1 minute to 2 hours, or 5 minutes to 60 minutes or about 10 minutes. The stabilized ACP and/or ACFP may be repeatedly applied to the dental surface over a period of 1 day to several months.

In one embodiment, calcium monofluorophosphate is contacted with the dental surface 1 to 60 minutes, or 1 to 30 minutes, or 1 to 5 minutes prior to contacting the dental surface with the stabilized ACP and/or ACFP.

In one embodiment, calcium monofluorophosphate is contacted with the dental surface 1 to 60 minutes, or 1 to 30 minutes, or 1 to 5 minutes after contacting the dental surface with the stabilized ACP and/or ACFP.

In a further aspect of the present invention there is provided a method of mineralizing a tooth surface comprising applying a stabilized ACP and/or ACFP complex and calcium monofluorophosphate to a tooth surface. Preferably the tooth surface is tooth enamel or dentine. Typically, the tooth surface is tooth enamel containing a lesion selected from the group consisting of one or more of a white spot lesion; a fluorotic lesion; a caries lesion; or a lesion caused by tooth erosion. Preferably, the stabilized ACP and/or ACFP complex and calcium monofluorophosphate are contained in the same composition that is applied to the tooth surface.

The present invention also provides a method of mineralizing a dental surface or sub-surface comprising contacting the dental surface or subsurface with a compound containing monofluorophosphate, a calcium source and a mineralizing agent, wherein the compound containing monofluorophosphate and a calcium source are provided to the oral cavity in conditions allowing the formation of calcium monofluorophosphate. Preferably, the compound containing monofluorophosphate is as described herein, even more preferably the compound containing monofluorophosphate is disodium monofluorophosphate. Preferably, the calcium source is as described herein, even more preferably the calcium source is calcium chloride.

The present invention also provides a method of mineralizing a dental surface or sub-surface comprising providing a composition to contact the dental surface or subsurface, wherein the composition, prior to contacting the dental surface or subsurface, comprises calcium monofluorophosphate and a mineralizing agent. In other words, the calcium monofluorophosphate is present in the composition before the composition contacts the dental surface or subsurface or before administration to the oral cavity. Preferably, the mineralizing agent is phosphopeptide stabilized ACP and/or ACFP, such as CPP-ACP and/or CPP-ACFP.

In any method or composition of the invention, the calcium monofluorophosphate may have been formed by a method comprising the step of:

mixing a source of monofluorophosphate and a source of calcium at a pH between about 5.0 to about 7.0,

thereby forming calcium monofluorophosphate.

Preferably, the source of monofluorophosphate is as described herein, more preferably the source of monofluorophosphate is disodium monofluorophosphate. Preferably, the source of calcium is as described herein, more preferably the source of calcium is calcium chloride. Typically, the pH is about 7.0.

The source of monofluorophosphate and source of calcium may be mixed at about equivalent amounts. For example, about 80 μmol of disodium monofluorophosphate per g may be mixed thoroughly with 80.0 μmol of calcium chloride (CaCl₂) per g. In one embodiment, 76.3 μmol of disodium monofluorophosphate per g is mixed thoroughly with 80.0 μmol of calcium chloride (CaCl₂) per g.

In any method or composition of the invention, the calcium monofluorophosphate may be formed by the method described in Example 9.

In any method or composition of the invention, ACP or ACFP is stabilised by CPP. Preferably, the CPP-ACP complex consists essentially of phosphopeptides, calcium, phosphate and hydroxide ions and water. Preferably, the CPP-ACFP complex consists essentially of phosphopeptides, calcium, phosphate, fluoride and hydroxide ions and water.

In any embodiment, the dental surface is in need of such treatment. Therefore the invention includes in addition to the steps of any method described herein a step of identifying a subject suffering fluorosis, dental caries, dentinal hypersensitivity or dental calculus, a white spot lesion; a fluorotic lesion; a caries lesion; or a lesion caused by tooth erosion.

The present invention provides a composition for mineralizing a dental surface or sub-surface comprising calcium monofluorophosphate and stabilized amorphous calcium phosphate (ACP) and/or amorphous calcium fluoride phosphate (ACFP).

The present invention provides a composition comprising calcium monofluorophosphate and stabilized amorphous calcium phosphate (ACP) and/or amorphous calcium fluoride phosphate (ACFP) for use in mineralizing a dental surface or sub-surface.

In a further aspect, there is provided a method of treating or preventing one or more of each of dental caries, tooth decay, dental erosion and fluorosis, comprising the steps of administering calcium monofluorophosphate to the teeth of a subject followed by administering an ACP or ACFP complex or composition. Topical administration of the complex is preferred. The method preferably includes the administration of the complex in a formulation as described herein.

In a further aspect there is provided the use of calcium monofluorophosphate and a stabilized amorphous calcium phosphate (ACP) or amorphous calcium fluoride phosphate (ACFP) in the manufacture of a composition for the treatment and/or prevention of one or more of dental caries, tooth decay, dental erosion and fluorosis.

In a further aspect there is provided a composition comprising as an active agent calcium monofluorophosphate and a stabilized amorphous calcium phosphate (ACP) or amorphous calcium fluoride phosphate (ACFP) for mineralizing a dental surface or sub-surface. Typically, mineralizing a dental surface or subsurface is for the treatment and/or prevention of one or more of dental caries, tooth decay, dental erosion and fluorosis.

In a further aspect or, in any method or use of the invention above, there is further provided a step of applying a source of free fluoride ions. The source of free fluoride ions may be applied simultaneously as calcium monofluorophosphate and the source of ACP or ACFP. Alternatively, the source of free fluoride ions may be applied prior to, or after, calcium monofluorophosphate or the source of ACP or ACFP. Exemplary sources of free fluoride ions include sodium fluoride, stannous fluoride and amine fluoride. Without being bound by any theory, these free fluoride ions may interact with ACP or ACFP to form fluorapatite on contact with the tooth surface, which may be more resistant to acid challenge than normal tooth enamel.

The present invention also provides a composition comprising calcium monofluorophosphate and a mineralizing agent. Preferably, the mineralizing agent is stabilized-amorphous calcium phosphate (ACP) or amorphous calcium fluoride phosphate (ACFP). Preferably, the composition further includes a pharmaceutically acceptable carrier, diluent or excipient.

In a preferred embodiment, the phosphopeptide stabilized amorphous calcium phosphate (ACP) or amorphous calcium fluoride phosphate (ACFP) complex in the composition has tightly bound and loosely bound calcium, wherein the bound calcium in the complex is less than the tightly bound calcium in an ACP or ACFP complex formed at a pH of 7.0. Optionally, the ACP or ACFP is predominantly in a basic form.

In another preferred embodiment, the calcium ion content of the stabilized ACP or ACFP complex in the composition is in the range of about 30 to 100 moles of calcium per mole of PP. More preferably, the calcium ion content is in the range of about 30 to about 50 moles of calcium per mole of PP.

In any embodiment, the ACP and/or ACFP in the composition can be in the form of a casein phosphopeptide stabilized ACP and/or ACFP complex.

The invention also relates to a kit for the treatment or prevention of one or more of dental caries, fluorosis and dental erosion including (a) calcium monofluorophosphate, and (b) a stabilized-ACP and/or stabilized-ACFP complex in a pharmaceutically acceptable carrier. Desirably, the kit further includes instructions for their use for the mineralization of a dental surface in a patient in need of such treatment. The instructions may describe the use of the kit to treat or prevent one or more of each of dental caries, tooth decay, dental erosion and fluorosis. In one embodiment, the agent and the complex are present in suitable amounts for treatment of a patient. Preferably, the stabilized ACP and/or ACFP is phosphopeptide (PP)-stabilized. Preferably, the phosphopeptide (as defined below) is a casein phosphopeptide. Preferably, the ACP or ACFP is in the form of a casein phosphopeptide stabilized ACP or ACFP complex.

The composition or kit of the invention may further include a source of free fluoride ions. The free fluoride ions may be from any suitable source. A source of free fluoride ions may include free fluoride ions or fluoride salts. Examples of sources of fluoride ions include, but are not limited to the following: sodium fluoride, stannous fluoride, sodium silicofluoride, silver fluoride, amine fluoride or any metal ion fluoride salt. A source of fluoride ions may be a hypofluorite. These source of fluoride ions may be provided in solution (typically an aqueous solution), or a suspension.

### BRIEF DESCRIPTION OF THE DRAWINGS

FIG. 1: The appliances prepared as described in Example 8 used in the clinical trial described in Example 10.

FIG. 2: Remineralisation of enamel subsurface lesions in situ by sodium fluoride (NaF) compared with calcium monofluorophosphate (CaMFP) with and without CPP-ACP. Upper appliance results shown in columns on the left, lower appliance results shown in columns on the right. The compositions containing calcium monofluorophosphate resulted in statistically significantly greater remineralisation than those containing sodium fluoride. * Statistically analysis by Repeated Measures Analysis of Variance and the level of significance was p<0.05.

### DETAILED DESCRIPTION OF THE INVENTION

Further aspects of the present invention and further embodiments of the aspects described in the preceding paragraphs will become apparent from the following description, given by way of example and with reference to the accompanying drawings. It will be understood that the invention disclosed and defined in this specification extends to all alternative combinations of two or more of the individual features mentioned or evident from the text or drawings. All of these different combinations constitute various alternative aspects of the invention.

Reference will now be made in detail to certain embodiments of the invention. While the invention will be described in conjunction with the embodiments, it will be understood that the intention is not to limit the invention to those embodiments. On the contrary, the invention is intended to cover all alternatives, modifications, and equivalents, which may be included within the scope of the present invention as defined by the claims.

One skilled in the art will recognize many methods and materials similar or equivalent to those described herein, which could be used in the practice of the present invention. The present invention is in no way limited to the methods and materials described. All of the patents and publications referred to herein are incorporated by reference in their entirety.

For purposes of interpreting this specification, terms used in the singular will also include the plural and vice versa. As used herein, except where the context requires otherwise, the term “comprise” and variations of the term, such as “comprising”, “comprises” and “comprised”, are not intended to exclude further additives, components, integers or steps. As used herein, except where the context requires otherwise, “comprise” and “include” can be used interchangeably.

The present invention provides a method of mineralizing a dental surface or sub-surface comprising contacting the dental surface or subsurface with calcium monofluorophosphate and a mineralizing agent. A dental subsurface is typically a hypomineralized lesion such that the compound and mineralizing agent contacted to the dental surface migrates through any surface layer, i.e. pellicle and/or plaque, through the porous dental surface to the region requiring mineralization. Preferably, the mineralizing agent is stabilized amorphous calcium phosphate (ACP) and/or amorphous calcium fluoride phosphate (ACFP). The dental surface is preferably dental enamel. The dental surface may be a lesion in the enamel, such as a lesion caused by caries, dental erosion or fluorosis.

Unless expressly indicated otherwise, reference herein to calcium monofluorophosphate also includes compounds or compositions that contain monofluorophosphate and calcium or that could produce calcium monofluorphosphate when exposed to an aqueous solution. In other words, in any method or any composition of the invention, the calcium monofluorophosphate may be formed prior to administration to an oral cavity, or the calcium monofluorophosphate may be formed in the oral cavity by a compound or source of monofluorophosphate, for example disodium monofluorophosphate, and a compound or source of calcium, for example calcium chloride. Preferred compounds that provide, or allow formation of, calcium monofluorophosphate include sodium monofluorophosphate plus a calcium source (e.g. a calcium salt). Sodium monofluorophosphate plus a calcium source (e.g. a calcium salt) may form calcium monofluorophosphate when exposed to the appropriate conditions, such as the amounts and pH, described below and described in the Examples. Calcium monofluorophosphate will not form simply by adding CPP-ACP and sodium monofluorophosphate as the ACP is already in a stabilised form. Further calcium monofluorophosphate will not form from sodium monofluorophosphate mixed with water-insoluble polishing materials commonly found in toothpastes such as tricalcium phosphate, dihydrated calcium phosphate, anhydrous dicalcium phosphate or calcium pyrophosphate. In addition, there is not enough calcium present in the oral cavity, let alone in a bioavailable form, to generate a therapeutically effective amount of calcium monofluorophosphate when, for example, sodium monofluorophosphate or any other monofluorphosphate containing compound is present in the oral cavity. While some calcium exists in the oral cavity this is only a fraction of the calcium required to form a therapeutically effective amount of calcium monofluorophosphate as described herein.

The present invention unexpectedly identifies that the neutral ion pair CaFPO₃⁰ is superior to F⁻ in promoting enamel remineralisation. This was unexpected as it was previously thought that free fluoride ions were required for remineralization of a surface of subsurface lesion and it was believed that monofluorophosphate particularly calcium monofluorophosphate would not liberate free fluoride ions. Therefore, monofluorophosphate was not considered an appropriate compound for use in remineralization of a surface or subsurface lesions, particularly in conjunction with phosphopeptide stabilized-ACP and/or ACFP. Without being bound by any theory or mode of action it is believed that monofluorophosphate forms a neutral ion pair with calcium ions Ca²⁺+FPO₃²⁻⇄CaFPO₃⁰ and this neutral ion pair facilitates the production of fluorapatite in the surface or subsurface lesion. Calcium monofluorophosphate is not readily hydrolysed intra-orally, outside the lesion, unlike sodium monofluorophosphate, This is advantageous as calcium monofluorophosphate is maintained as a neutral ion thereby more easily entering the lesion, whereas sodium monofluorophosphate dissociates to form negative ions e.g. PFO₃²⁻ or HPFO₃¹⁻, and negative ions are attracted/repelled by the enamel/plaque charged surface restricting their entry into the lesion. The neutral ion pair CaFPO₃⁰ can diffuse down concentration gradients into the subsurface enamel lesion and effect remineralisation by providing calcium, fluoride and phosphate ions. CaFPO₃⁰ is superior to the neutral ion pair CaHPO₄⁰ as it provides Ca, F and phosphate whereas the other neutral ion pair only provides calcium and phosphate ions.

In the field of the invention, the amount of fluoride in a composition is usually described in parts per million (ppm). A skilled person in this field therefore refers to the fluoride content by reference to the ppm of fluoride that is released by the fluoride containing compound such as monofluorophosphate. Of physiological importance is bioavailable fluoride, i.e. the amount of fluoride it contains and releases. In any aspect of the invention, the calcium monofluorophosphate is present in an amount outlined below. For example, the calcium monofluorophosphate may be present in a composition of the invention or used in a method of the invention of about 200 ppm to 5000 ppm F. In a preferred embodiment, calcium monofluorophosphate is present in an amount of about 200 ppm to about 3000 ppm F, 400 ppm to about 1500 ppm F, or about 1000 ppm to 1450 ppm F. In a further preferred embodiment, calcium monofluorophosphate is present in an amount of about above 500, 900, 1000, 1100, 1200, 1300, 1400 or 1500 ppm F.

In any composition or method of the invention, the calcium monofluorophosphate is present in greater than about 1 mM, 5 mM, 10 mM, 20 mM, 40 mM, 50 mM, 60 mM or 70 mM. Preferably, the calcium monofluorophosphate is present in about 1 mM, 5 mM, 10 mM, 20 mM, 40 mM, 50 mM, 60 mM, 70 mM or 75 mM.

Any composition or method of the invention comprises calcium monofluorophosphate in an amount that when contacted to a dental surface or subsurface results in a clinically observable amount of remineralization compared to a when calcium monofluorophosphate is not present.

In any aspect of the invention described herein, reference to a compound that provides monofluorophosphate includes calcium monofluorophosphate.

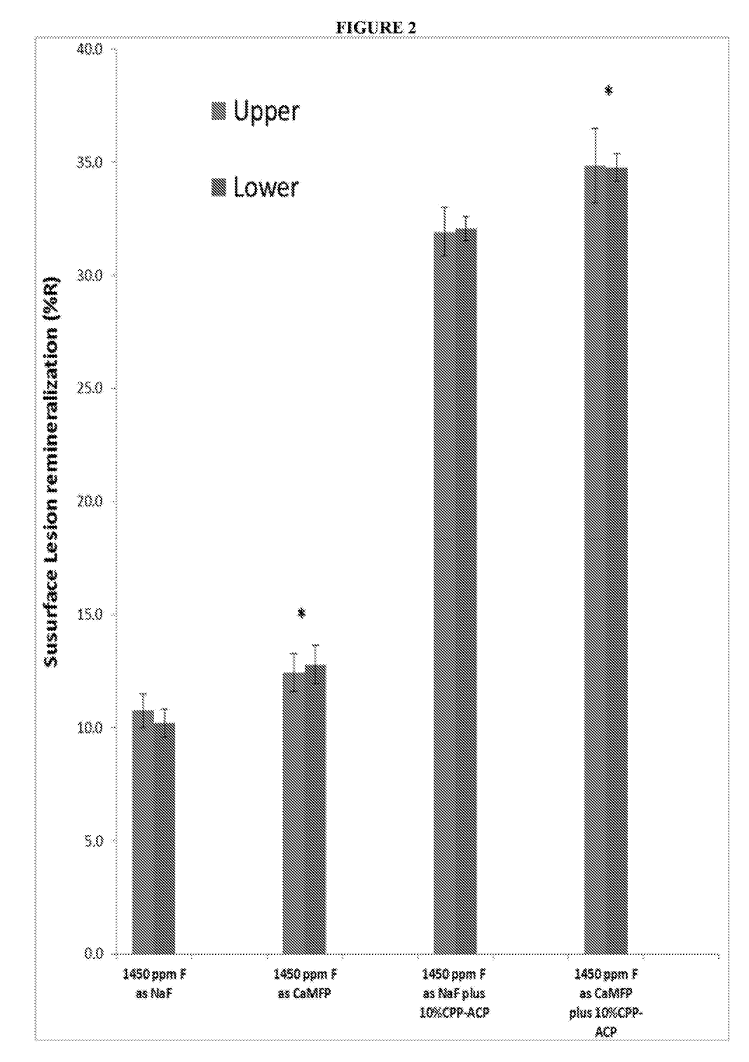

A stabilized-ACP or ACFP complex as described in the current specification is the “closed” complexes are shown in FIG. 2 of Cross et al., 2007. It has been shown by Cross et al. that the CPP-ACP complexes produced at pH 7 are “open”, whereas CPP-ACP produced with the continuous addition of hydroxide ions are “closed” complexes. This “closed” complex is significantly different from the “open” complex as it has a completely different structure and contains basic calcium phosphate phases. Clinically it has been shown to have substantially improved effect and delivered more calcium phosphate to teeth.

A stabilized-ACP or ACFP complex as referred to herein includes a stabilized-ACP or ACFP complex as described in WO2006/056013 the contents of which are incorporated by reference.

In a preferred embodiment, the phosphopeptide stabilized amorphous calcium phosphate (ACP) or amorphous calcium fluoride phosphate (ACFP) complex has tightly bound and loosely bound calcium, wherein the bound calcium in the complex is less than the tightly bound calcium in an ACP or ACFP complex formed at a pH of 7.0. Optionally, the ACP or ACFP is predominantly in a basic form.

A stabilized-ACP or ACFP complex as referred to herein include a stabilized-ACP or ACFP complex formed at a pH of below 7.0. Preferably the complex is formed at a pH in the range of about 5.0 up to but below 7.0. More preferably the complex is formed at a pH range of about 5.0 to about 6.0. In a preferred embodiment, the complex is formed at a pH of about 5.0 or about 5.5. Preferably, the ACP or ACFP in the complex is predominantly in a basic form.

A stabilized-ACP may be produced by a method comprising the steps of:

(i) obtaining a solution comprising at least one phosphopeptide and;

(ii) admixing solutions comprising calcium ions, phosphate ions and hydroxide ions, while maintaining the pH at about 7.0 or below.

A stabilized-ACFP may be produced by a method comprising the steps of:

(i) obtaining a solution comprising at least one phosphopeptide and;

(ii) admixing solutions comprising calcium ions, phosphate ions, hydroxide ions and fluoride ions, while maintaining the pH at about 7.0 or below.

The hydroxide ions may be titrated into the solution to maintain the phosphopeptide solution at an essentially constant pH. The calcium and phosphate ions may be titrated into the phosphopeptide solution with constant mixing and at a rate that avoids the formation of a calcium phosphate precipitate in the phosphopeptide solution.

As used herein a phosphopeptide stabilized amorphous calcium phosphate (ACP) or amorphous calcium fluoride phosphate (ACFP) complex may also include ACP in the complex that has tightly bound and loosely calcium, wherein the tightly bound calcium in the complex is less than the tightly bound calcium in an ACP or ACFP complex formed at a pH of 7.0 and the ACP or ACFP is predominantly in a basic form, obtainable or obtained by a method comprising:

a) admixing a first solution comprising calcium ions, a second solution comprising phosphate ions, and optionally a third solution comprising fluoride ions, to a solution comprising phosphopeptides and a solvent with a pH of from about 5 up to but below 7; and

b) maintaining the pH of the solution at about 5.0 up to but below 7.0 during the admixing by adding hydroxide ions.

“Tightly” and “loosely” bound calcium and phosphate can be determined using analytical ultrafiltration as shown in Example 2. Briefly, the solution of phosphopeptide, calcium, phosphate and optionally fluoride admixed while maintaining the pH at about 7.0 or below can be first filtered through a 0.1 micron filter to remove free calcium and phosphate that is not associated with the complexes. This free calcium and phosphate is present in the filtrate and discarded. Any free calcium or phosphate that is not associated in any way with the complexes would not be bioavailable, i.e. delivered by the phosphopeptide to the tooth. The retentate from the 0.1 micron filtration can be further analyzed by centrifugation through a 3000 mw cutoff filter at 1,000 g for 15 min. The resulting filtrate contains calcium and phosphate that is loosely bound or associated with the complexes. At this centrifugal force calcium and phosphate that is not tightly bound to the complexes are released and move to into the filtrate. The Ca and Pi that is tightly bound in the complexes is retained in the retentate. The amount of tightly bound Ca and Pi in the retentate can then be determined by subtracting the amount of Ca and Pi in the filtrate from the total amount of Ca and Pi in the retentate of the 0.1 micron filtration.

A stabilized-ACP or ACFP complex as referred to herein include a stabilized-ACP or ACFP complex as described in WO2006/135982 the contents of which are incorporated by reference.

A “superloaded” phosphopeptide or phosphoprotein (PP) stabilized-amorphous calcium phosphate (ACP) or amorphous calcium fluoride phosphate (ACFP) complex. The complex may be formed at any pH (eg 3-10). Preferably the phosphopeptide includes the sequence -A-B-C-, where A is a phosphoamino acid, preferably phosphoserine, B is any amino acid including a phosphoamino acid and C is glutamic acid, aspartic acid or a phosphoamino acid. The phosphoamino acid may be phosphoserine. The PP is superloaded with calcium and phosphate ions. The calcium ions may be in the range 30-1000 mol Ca per mole of PP, or in the range of 30-100 or 30-50 mole Ca per mole of PP. In another embodiment, the mol Ca per mol of PP is at least 25, 30, 35, 40, 45 or 50.

The present invention includes a phosphopeptide or phosphoprotein (PP) stabilized amorphous calcium phosphate or amorphous calcium fluoride phosphate complex having a calcium ion content greater than about 30 moles of calcium per mole of PP. In a preferred embodiment, the calcium ion content is in the range of about 30 to 100 moles of calcium per mole of PP. More preferably, the calcium ion content is in the range of about 30 to about 50 moles of calcium per mole of PP.

The invention also provides a phosphopeptide or phosphoprotein (PP) stabilized-amorphous calcium phosphate (ACP) or amorphous calcium fluoride phosphate (ACFP) complex produced by a method comprising the steps of:

(i) obtaining solutions comprising calcium, inorganic phosphate and fluoride (optional); and

(ii) admixing (i) with a solution comprising PP-ACP.

In a preferred embodiment, the PP is casein phosphopeptide (CPP).

In a further aspect, the present invention also includes use of a formulation of (a) a PP stabilized ACP and/or ACFP complex together with at least an equal amount by weight of calcium phosphate, and (b) a compound that provides monofluorophosphate. Preferably the calcium phosphate is CaHPO₄ or calcium lactate or any other soluble calcium phosphate compound. Preferably, the calcium phosphate (e.g. CaHPO₄) is dry blended with the PP stabilized ACP and/or ACFP complex. In a preferred embodiment, the PP-ACP and/or PP-ACFP complex:calcium phosphate ratio is about 1:1-50. more preferably about 1: 1-25, more preferably about 1:5-15. In one embodiment, the PP-ACP and/or PP-ACFP complex:calcium phosphate ratio is about 1:10.

The oral care formulation that includes a phosphopeptide or phosphoprotein (PP) stabilized amorphous calcium phosphate (ACP) and/or amorphous calcium fluoride phosphate (ACFP) complex having a calcium ion content greater than about 30 moles of calcium per mole of PP when used in the oral cavity may be produced by a method including the steps of:

(i) obtaining a powder including a PP-ACP and/or PP-ACFP complex;

(ii) dry blending with an effective amount of calcium phosphate and a compound that provides calcium monofluorophosphate; and

(iii) formulating the dry blended mixture into an oral care formulation.

Preferably, the form of calcium phosphate for dry blending is any soluble calcium phosphate including, but not limited to, CaHPO₄, Ca₂HPO₄ and calcium lactate.

A stabilized-ACP or ACFP complex as referred to herein may also include a stannous-associated stabilized amorphous calcium phosphate (ACP) and/or amorphous calcium fluoride phosphate (ACFP) complex as described in the PCT application PCT/AU2014/050447 filed on 24 Dec. 2014 in the name of The University of Melbourne. The stannous may be bound to the stabilized amorphous calcium phosphate (ACP) and/or amorphous calcium fluoride phosphate (ACFP) as determined using the experimental protocol in Example 2. In one embodiment, stannous-associated stabilized amorphous calcium phosphate (ACP) and/or amorphous calcium fluoride phosphate (ACFP) complex are produced by the method as described herein, including but not limited to the method described in Example 1.

Casein phosphopeptides, can stabilize a stannous compound in an aqueous environment and in the presence of stabilized amorphous calcium phosphate (ACP) and stabilized amorphous calcium fluoride phosphate (ACFP) these complexes are superior to other forms of fluoride and stabilized ACP or ACFP in remineralizing enamel subsurface lesions. Mineralization of dental surfaces can be significantly enhanced by providing a stannous compound during the process of mineralization by stabilized ACP and/or stabilized ACFP. In particular, it has been found that the mineralization of enamel by stabilized soluble forms of stannous-associated ACP complexes and stannous-associated ACFP complexes is enhanced compared with stabilized ACP and fluoride without associated stannous. In other words, the stannous ions complex with CPP-ACP and/or CPP-ACFP complexes, and these Sn-associated CPP-AC(F)P complexes then deliver superior properties. Various compositions incorporating these complexes for administration are useful. Where the fluoride stannous salt is used, additional fluoride ions are available in compositions of the stannous-associated ACP/ACFP complexes. Additional fluoride ions may also be provided by inclusion of NaF in the composition.

A stannous-associated stabilized ACP or ACFP may have a stannous ion content of at least 1 mole of stannous per mole of phosphopeptide. Preferably, the stannous-associated stabilized ACP or ACFP has a stannous ion content of at least 2, 3, 4, 5, 6, 7, 8, 9 or 10 moles of stannous per mole of phosphopeptide. Even more preferably, the stannous ion content is in the range of 1 to 100, 1 to 50, 1 to 20 or 1 to 10 moles of stannous per mole of phosphopeptide.

A stannous-associated stabilized ACP or ACFP may be prepared by the following. Casein phosphopeptide-amorphous calcium phosphate (CPP-ACP) is acquired from Cadbury Enterprises Pte Ltd under the trademark name Recaldent™. A solution is prepared using CPP-ACP, SnF2 and NaF to produce at 0.4% w/v CPP-ACP, 220 ppm F as SnF2 and 70 ppm F as NaF, pH 5.6. Specifically, the stannous-associated stabilized ACP complexes may be prepared by adding CPP-ACP to distilled/deionised water and then SnF2 (solid) and NaF added with addition of 1 M HCl to maintain the pH between 4.0-6.5. The pH may not be allowed to go above 6.5. The total volume of acid added was less than 1% of the stannous-associated stabilized ACP solution volume. While NaF may be added in this method it is a minor component and the majority of the fluoride derives from the SnF2. The method could be performed using SnF2 only (without NaF).

The present invention also provides a method of mineralizing a dental surface or sub-surface including the steps of:

(i) contacting the dental surface with a protein disrupting agent, and

(ii) contacting the dental surface with a composition of the invention. The dental surface is preferably dental enamel. In one embodiment the dental surface is a lesion in the enamel, such as a lesion caused by caries, dental erosion or fluorosis. Any suitable protein disrupting agent can be used in the method of the present invention. The agent is required to reduce the proteinaceous barrier formed over the surface to be treated, such as the pellicle over teeth. Examples of suitable agents include bleach, detergent, chaotropic agents such as urea, high phosphate concentrations, cocktails of proteases (e.g. endopeptidases, proteinases and exopeptidases) and any other protein solubilizing, disrupting or hydrolysing agent. Examples of suitable bleaches include sodium hypochlorite (NaOCl), and cabamide peroxide bleaches. In a preferred embodiment, the bleach is an alkaline bleach. In a further preferred embodiment the alkaline bleach is NaOCl. The protein disrupting agent acts to solubilize and partially or wholly remove proteins from the dental surface, particularly proteins of the pellicle.

A composition as described herein may further include free fluoride ions. The free fluoride ions may be from any suitable source. A source of free fluoride ions may include free fluoride ions or fluoride salts. Examples of sources of fluoride ions include, but are not limited to the following: sodium fluoride, stannous fluoride, sodium silicofluoride and amine fluoride. These may be provided in solution (typically an aqueous solution), or a suspension.

The free fluoride ions are preferably present in the composition in an amount greater than 1 ppm. More preferably, the amount is more than 3 ppm. In another embodiment, it is preferably more than 10 ppm. In typical embodiments described below, the amount may be several hundred or thousand ppm. The free fluoride content is typically measured as a ppm in oral compositions in the manner commonly used in the art. Where the fluoride is provided from a source with the stabilized ACP, the ppm refers to the concentration of the fluoride in that source, typically a solution or suspension of bioavailable fluoride.

Mineralization of dental surfaces can be significantly enhanced by increasing the pH of a lesion during the process of mineralization. In particular, it has been found that the mineralization of enamel by stabilized soluble forms of ACP (CPP-ACP) and ACFP (CPP-ACFP) is enhanced by a compound that increases the intra-lesion pH if the intra-lesion pH is acidic or maintains the intra-lesion pH if the intra-lesion pH is neutral or basic. For example, during the development of caries, the pH of the intra-lesion fluid may be 6 or below.

The compound which is capable of increasing or maintaining the pH of a solution may be contacted with the dental surface for a period of about 1 to 60 minutes, or for about 1 to 30 minutes. In one embodiment, the compound which is capable of increasing or maintaining the pH of a solution is contacted with the dental surface for about 20 minutes. An example of how this is achieved is formulating the compound into an oral composition, such as a paste, and then contacting or applying the composition to the dental surface. The oral composition, such as a paste, has sufficient viscosity to be retained on the tooth for the required time period.

Preferably the stabilized ACP and/or ACFP is contacted with the dental surface for a period of about 1 minute to 2 hours, or 5 minutes to 60 minutes or about 10 minutes. The stabilized ACP and/or ACFP may be repeatedly applied to the dental surface over a period of 1 day to several months.

In one embodiment, the compound which is capable of increasing or maintaining the pH of a solution is contacted with the dental surface 1 to 60 minutes, or 1 to 30 minutes, or 1 to 5 minutes prior to contacting the dental surface with the stabilized ACP and/or ACFP.

In one embodiment, the compound which is capable of increasing or maintaining the pH of a solution is contacted with the dental surface 1 to 60 minutes, or 1 to 30 minutes, or 1 to 5 minutes after contacting the dental surface with the stabilized ACP and/or ACFP.

In a further aspect of the present invention there is provided a method for mineralizing a tooth surface comprising applying an ACP and/or ACFP complex, a compound that provides monofluorophosphate and a compound which is capable of increasing or maintaining the pH of a solution to a tooth surface. Preferably the tooth surface is tooth enamel. Typically, the tooth surface is tooth enamel or dentine containing a lesion selected from the group consisting of one or more of a white spot lesion; a fluorotic lesion; a caries lesion; or a lesion caused by tooth erosion.

In one embodiment, the dental surface is in need of such treatment. Therefore, in another aspect, the invention includes in addition to the steps of any method described herein a step of identifying a subject suffering fluorosis, dental caries, dentinal hypersensitivity or dental calculus, a white spot lesion; a fluorotic lesion; a caries lesion; or a lesion caused by tooth erosion.

The present invention provides a composition comprising:

(a) a compound that is capable of increasing or maintaining the pH of a solution;

(b) stabilized ACP and/or ACFP; and

(c) a compound that provides monofluorophosphate

for use in mineralizing a dental surface or sub-surface.

A compound which is capable of increasing or maintaining the pH of a solution includes a compound which can accept hydrogen cations (protons) or, more generally, donate a pair of valence electrons. Preferably, the compound may commonly be a base. The compound is capable of increasing the pH of a solution that has an acidic pH (i.e. less than pH 7). Preferably, the compound is capable of raising the pH of intra-lesion fluid of a dental lesion from 6 to 7.5. In one embodiment, a compound which is capable of increasing or maintaining the pH of a solution is an alkali which has the capacity to release hydroxide ions.

A compound which is capable of increasing or maintaining the pH of a solution also includes a compound that can maintain as a buffer the pH of a neutral or basic solution (i.e. pH greater than or equal to 7) when the neutral or basic solution is exposed to an acid. Typically, the compound is capable of maintaining the pH of a solution between 7 to 9, preferably about 7.5.

Any pharmaceutically acceptable compounds described as a base are suitable for use in the invention. Typically, the base is suitable for oral use. Preferably, the compound acts as a base, i.e. only releases hydroxide ions or donates electrons, in the presence of an acid. The base may be a free-base form, or in a pharmaceutically acceptable salt form. Non-limiting examples of bases suitable for use in the invention include hydroxides, chlorides, borates, phosphates including hydrogen phosphates and dihydrogen phosphates, citrates, carbonates, bicarbonates, hypochlorites, amines and any salt forms thereof including an alkali metal salt forms. More specifically, non-limiting examples of suitable pharmaceutically acceptable bases include ammonium hydroxide, sodium hydroxide, potassium hydroxide, lithium hydroxide, calcium hydroxide, magnesium hydroxide, ferrous hydroxide, zinc hydroxide, copper hydroxide, aluminum hydroxide, ferric hydroxide, isopropylamine, trimethylamine, diethylamine, triethylamine, tripropylamine, ethanolamine, 2-dimethylaminoethanol, 2-diethylaminoethanol, lysine, arginine, histidine. A hypofluorite capable of acting as a base as described herein is also useful in the invention as the agent for increasing or maintaining pH. A suitable hypofluorite would react in situ to produce fluoride ions and hydroxide (or another base) ions. One skilled in the art will appreciate that fluoride ions can substitute for hydroxide in the crystal structure of apatite forming fluorapatite.

In any aspect or embodiments as described herein, the stabilized ACP and/or ACFP is phosphopeptide (PP)-stabilized. Preferably, the phosphopeptide (as defined below) is a casein phosphopeptide. Preferably, the ACP or ACFP is in the form of a casein phosphopeptide stabilized ACP or ACFP complex.

“Phosphopeptide” in the context of the description of this invention means an amino acid sequence in which at least one amino acid is phosphorylated. Preferably, the phosphopeptide includes one or more of the amino acid sequence -A-B-C-, where A is a phosphoamino residue, B is any amino acyl residue including a phosphoamino residue and C is selected from a glutamyl, aspartyl or phosphoamino residue. Any of the phosphoamino residues may independently be a phosphoseryl residue. B is desirably a residue the side-chain of which is neither relatively large nor hydrophobic. It may be Gly, Ala, Val, Met, Leu, Ile, Ser, Thr, Cys, Asp, Glu, Asn, Gln or Lys. Preferably at least two of the phosphoamino acids in the sequence are preferably contiguous. Preferably the phosphopeptide includes the sequence A-B-C-D-E, where A, B, C, D and E are independently phosphoserine, phosphothreonine, phosphotyrosine, phosphohistidine, glutamic acid or aspartic acid, and at least two, preferably three, of the A, B, C, D and E are a phosphoamino acid. In a preferred embodiment, the phosphoamino acid residues are phosphoserine, most preferably three contiguous phosphoserine residues. It is also preferred that D and E are independently glutamic or aspartic acid.

In one embodiment, the ACP or ACFP is stabilized by a casein phosphopeptide (CPP), which is in the form of intact casein or fragment of the casein, and the complex formed preferably has the formula [CPP(ACP)₈]ₙ or [(CPP)(ACFP)₈]ₙ where n is equal to or greater than 1, for example 6. The complex formed may be a colloidal complex, where the core particles aggregate to form large (eg 100 nm) colloidal particles suspended in water. Thus, the PP can be a casein protein or a phosphopeptide.

The PP may be from any source; it may be present in the context of a larger polypeptide, including a full length casein polypeptide, or it may be isolated by tryptic or other enzymatic or chemical digestion of casein, or other phosphoamino acid rich proteins such as phosphitin, or by chemical or recombinant synthesis, provided that it comprises the sequence -A-B-C- or A-B-C-D-E as described above. The sequence flanking this core sequence may be any sequence. However, those flanking sequences in αₛ₁(59-79), β(1-25), αₛ₂(46-70) and αₛ₂(1-21) are preferred. The flanking sequences may optionally be modified by deletion, addition or conservative substitution of one or more residues. The amino acid composition and sequence of the flanking region are not critical.

The phosphopeptide may be selected from any described in WO2006/056013, WO2006/135982 or U.S. Pat. No. 5,015,628.

Examples of conservative substitutions are shown in Table 1 below.

TABLE 1

| | Exemplary Conservative | Preferred Conservative |
|---|---|---|
| Original Residue | Substitution | Substitution |
| Ala | Val, Leu, Ile | Val |
| Asn | Gln Lys His Phe | Gln |
| Gln | Asn | Asn |
| Gly | Pro | Pro |
| Ile | Leu, Val, Met, Ala, Phe | Leu |
| Leu | Ile, Val, Met, Ala, Phe | Ile |
| Lys | Arg, Gln, Asn | Arg |
| Phe | Leu, Val, Ile, Ala | Leu |
| Pro | Gly | Gly |
| Ser | Thr | Thr |
| Val | Ile, Leu, Met, Phe, Ala | Leu |
| Asp | Glu | Glu |
| Thr | Ser | Ser |
| Trp | Tyr | Tyr |
| Tyr | Trp Phe Thr Ser | Phe |

The flanking sequences may also include non-naturally occurring amino acid residues. Commonly encountered amino acids which are not encoded by the genetic code, include:

2-amino adipic acid (Aad) for Glu and Asp;

2-aminopimelic acid (Apm) for Glu and Asp;

2-aminobutyric (Abu) acid for Met, Leu, and other aliphatic amino acids;

2-aminoheptanoic acid (Ahe) for Met, Leu and other aliphatic amino acids;

2-aminoisobutyric acid (Aib) for Gly;

cyclohexylalanine (Cha) for Val, and Leu and Ile;

homoarginine (Har) for Arg and Lys;

2, 3-diaminopropionic acid (Dpr) for Lys, Arg and His;

N-ethylglycine (EtGly) for Gly, Pro, and Ala;

N-ethylasparigine (EtAsn) for Asn, and Gln;

Hydroxyllysine (Hyl) for Lys;

allohydroxyllysine (AHyl) for Lys;

3-(and 4) hydroxyproline (3Hyp, 4Hyp) for Pro, Ser, and Thr;

alloisoleucine (Alle) for Ile, Leu, and Val;

p-amidinophenylalanine for Ala;

N-methylglycine (MeGly, sarcosine) for Gly, Pro, Ala.

N-methylisoleucine (MeIle) for Ile;

Norvaline (Nva) for Met and other aliphatic amino acids;

Norleucine (Nle) for Met and other aliphatic amino acids;

Ornithine (Orn) for Lys, Arg and His;

Citrulline (Cit) and methionine sulfoxide (MSO) for Thr, Asn and Gln;

N-methylphenylalanine (MePhe), trimethylphenylalanine, halo (F, Cl, Br and I) phenylalanine, triflourylphenylalanine, for Phe.

In one embodiment, the PP is one or more phosphopeptides selected from the group consisting of αₛ₁(59-79), β(1-25), αₛ₂(46-70) and αₛ₂(1-21).

In another embodiment of the invention, the stabilized ACFP or ACP complex and a compound that provides monfluorphosphate is incorporated into oral compositions such as toothpaste, mouth washes or formulations for the mouth to aid in the prevention and/or treatment of dental caries, tooth decay, dental erosion or fluorosis. The ACFP or ACP complex may comprise 0.01-50% by weight of the composition, preferably 1.0-50%. For oral compositions, it is preferred that the amount of the CPP-ACP and/or CPP-ACFP administered is 0.01-50% by weight, preferably 1.0%-50% by weight of the composition. In a particularly preferred embodiment, the oral composition of the present invention contains about 2% CPP-ACP, CPP-ACFP or a mixture of both. The oral composition of this invention which contains the above-mentioned agents may be prepared and used in various forms applicable to the mouth such as dentifrice including toothpastes, toothpowders and liquid dentifrices, mouthwashes, mouthrinses, mouth sprays, varnish, dental cement, troches, chewing gums, dental pastes, gingival massage creams, gargle tablets, dairy products and other foodstuffs. The oral composition according to this invention may further include additional well known ingredients depending on the type and form of a particular oral composition.

In certain preferred forms of the invention the oral composition may be substantially liquid in character, such as a mouthwash, rinse or spray. In such a preparation the vehicle is typically a water-alcohol mixture desirably including a humectant as described below. Generally, the weight ratio of water to alcohol is in the range of from about 1:1 to about 20:1. The total amount of water-alcohol mixture in this type of preparation is typically in the range of from about 70 to about 99.9% by weight of the preparation. The alcohol is typically ethanol or isopropanol. Ethanol is preferred.

In other desirable forms of this invention, the composition may be substantially solid or pasty in character, such as toothpowder, a dental tablet or a toothpaste (dental cream) or gel dentifrice. The vehicle of such solid or pasty oral preparations generally contains dentally acceptable polishing material. Examples of polishing materials are water-insoluble sodium metaphosphate, potassium metaphosphate, tricalcium phosphate, dihydrated calcium phosphate, anhydrous dicalcium phosphate, calcium pyrophosphate, magnesium orthophosphate, trimagnesium phosphate, calcium carbonate, hydrated alumina, calcined alumina, aluminium silicate, zirconium silicate, silica, bentonite, and mixtures thereof. Other suitable polishing material include the particulate thermosetting resins such as melamine-, phenolic, and urea-formaldehydes, and cross-linked polyepoxides and polyesters. Preferred polishing materials include crystalline silica having particle sizes of up to about 5 microns, a mean particle size of up to about 1.1 microns, and a surface area of up to about 50,000 cm²/g., silica gel or colloidal silica, and complex amorphous alkali metal aluminosilicate.

When visually clear gels are employed, a polishing agent of colloidal silica, such as those sold under the trademark SYLOID as Syloid 72 and Syloid 74 or under the trademark SANTOCEL as Santocel 100, alkali metal aluminosilicate complexes are particularly useful since they have refractive indices close to the refractive indices of gelling agent-liquid (including water and/or humectant) systems commonly used in dentifrices.

Many of the so-called “water insoluble” polishing materials are anionic in character and also include small amounts of soluble material. Thus, insoluble sodium metaphosphate may be formed in any suitable manner, for example as illustrated by Thorpe's Dictionary of Applied Chemistry, Volume 9, 4th Edition, pp. 510-511. The forms of insoluble sodium metaphosphate known as Madrell's salt and Kurrol's salt are further examples of suitable materials. These metaphosphate salts exhibit only a minute solubility in water, and therefore are commonly referred to as insoluble metaphosphates (IMP). There is present therein a minor amount of soluble phosphate material as impurities, usually a few percent such as up to 4% by weight. The amount of soluble phosphate material, which is believed to include a soluble sodium trimetaphosphate in the case of insoluble metaphosphate, may be reduced or eliminated by washing with water if desired. The insoluble alkali metal metaphosphate is typically employed in powder form of a particle size such that no more than 1% of the material is larger than 37 microns.

The polishing material is generally present in the solid or pasty compositions in weight concentrations of about 10% to about 99%. Preferably, it is present in amounts from about 10% to about 75% in toothpaste, and from about 70% to about 99% in toothpowder. In toothpastes, when the polishing material is silicious in nature, it is generally present in an amount of about 10-30% by weight. Other polishing materials are typically present in amount of about 30-75% by weight.

In a toothpaste, the liquid vehicle may comprise water and humectant typically in an amount ranging from about 10% to about 80% by weight of the preparation. Glycerine, propylene glycol, sorbitol and polypropylene glycol exemplify suitable humectants/carriers. Also advantageous are liquid mixtures of water, glycerine and sorbitol. In clear gels where the refractive index is an important consideration, about 2.5-30% w/w of water, 0 to about 70% w/w of glycerine and about 20-80% w/w of sorbitol are preferably employed.

Toothpaste, creams and gels typically contain a natural or synthetic thickener or gelling agent in proportions of about 0.1 to about 10, preferably about 0.5 to about 5% w/w. A suitable thickener is synthetic hectorite, a synthetic colloidal magnesium alkali metal silicate complex clay available for example as Laponite (e.g. CP, SP 2002, D) marketed by Laporte Industries Limited. Laponite D is, approximately by weight 58.00% SiO₂, 25.40% MgO, 3.05% Na₂O, 0.98% Li₂O, and some water and trace metals. Its true specific gravity is 2.53 and it has an apparent bulk density of 1.0 g/ml at 8% moisture.

Other suitable thickeners include Irish moss, iota carrageenan, gum tragacanth, starch, polyvinylpyrrolidone, hydroxyethylpropylcellulose, hydroxybutyl methyl cellulose, hydroxypropyl methyl cellulose, hydroxyethyl cellulose (e.g. available as Natrosol), sodium carboxymethyl cellulose, and colloidal silica such as finely ground Syloid (e.g. 244). Solubilizing agents may also be included such as humectant polyols such propylene glycol, dipropylene glycol and hexylene glycol, cellosolves such as methyl cellosolve and ethyl cellosolve, vegetable oils and waxes containing at least about 12 carbons in a straight chain such as olive oil, castor oil and petrolatum and esters such as amyl acetate, ethyl acetate and benzyl benzoate.

It will be understood that, as is conventional, the oral preparations will usually be sold or otherwise distributed in suitable labelled packages. Thus, a jar of mouth rinse will have a label describing it, in substance, as a mouth rinse or mouthwash and having directions for its use; and a toothpaste, cream or gel will usually be in a collapsible tube, typically aluminium, lined lead or plastic, or other squeeze, pump or pressurized dispenser for metering out the contents, having a label describing it, in substance, as a toothpaste, gel or dental cream.

Organic surface-active agents may be used in the compositions of the present invention to achieve increased prophylactic action, assist in achieving thorough and complete dispersion of the active agent throughout the oral cavity, and render the instant compositions more cosmetically acceptable. The organic surface-active material is preferably anionic, non-ionic or ampholytic in nature and preferably does not interact with the active agent. It is preferred to employ as the surface-active agent a detersive material which imparts to the composition detersive and foaming properties. Suitable examples of anionic surfactants are water-soluble salts of higher fatty acid monoglyceride monosulfates, such as the sodium salt of the monosulfated monoglyceride of hydrogenated coconut oil fatty acids, higher alkyl sulfates such as sodium lauryl sulfate, alkyl aryl sulfonates such as sodium dodecyl benzene sulfonate, higher alkylsulfo-acetates, higher fatty acid esters of 1,2-dihydroxy propane sulfonate, and the substantially saturated higher aliphatic acyl amides of lower aliphatic amino carboxylic acid compounds, such as those having 12 to 16 carbons in the fatty acid, alkyl or acyl radicals, and the like. Examples of the last mentioned amides are N-lauroyl sarcosine, and the sodium, potassium, and ethanolamine salts of N-lauroyl, N-myristoyl, or N-palmitoyl sarcosine which should be substantially free from soap or similar higher fatty acid material. The use of these sarconite compounds in the oral compositions of the present invention is particularly advantageous since these materials exhibit a prolonged marked effect in the inhibition of acid formation in the oral cavity due to carbohydrates breakdown in addition to exerting some reduction in the solubility of tooth enamel in acid solutions. Examples of water-soluble non-ionic surfactants suitable for use are condensation products of ethylene oxide with various reactive hydrogen-containing compounds reactive therewith having long hydrophobic chains (e.g. aliphatic chains of about 12 to 20 carbon atoms), which condensation products (“ethoxamers”) contain hydrophilic polyoxyethylene moieties, such as condensation products of poly (ethylene oxide) with fatty acids, fatty alcohols, fatty amides, polyhydric alcohols (e.g. sorbitan monostearate) and polypropyleneoxide (e.g. Pluronic materials).

The surface active agent is typically present in amount of about 0.1-5% by weight. It is noteworthy, that the surface active agent may assist in the dissolving of the active agent of the invention and thereby diminish the amount of solubilizing humectant needed.

Various other materials may be incorporated in the oral preparations of this invention such as whitening agents, preservatives, silicones, chlorophyll compounds and/or ammoniated material such as urea, diammonium phosphate, and mixtures thereof. These adjuvants, where present, are incorporated in the preparations in amounts which do not substantially adversely affect the properties and characteristics desired.

Any suitable flavouring or sweetening material may also be employed. Examples of suitable flavouring constituents are flavouring oils, e.g. oil of spearmint, peppermint, wintergreen, sassafras, clove, sage, eucalyptus, marjoram, cinnamon, lemon, and orange, and methyl salicylate. Suitable sweetening agents include sucrose, lactose, maltose, sorbitol, xylitol, sodium cyclamate, perillartine, AMP (aspartyl phenyl alanine, methyl ester), saccharine, and the like. Suitably, flavour and sweetening agents may each or together comprise from about 0.1% to 5% more of the preparation.

The compositions of this invention can also be incorporated in lozenges, or in chewing gum or other products, e.g. by stirring into a warm gum base or coating the outer surface of a gum base, illustrative of which are jelutong, rubber latex, vinylite resins, etc., desirably with conventional plasticizers or softeners, sugar or other sweeteners or such as glucose, sorbitol and the like. The composition of the invention may be a dual phase composition wherein each phase permits release of components over different time periods. For example, in use a dual phase composition may release stabilized ACP and/or ACFP, preferably CPP-ACP and/or CPP-ACFP, from a first phase at a faster rate than a compound that provides monofluorophosphate from a second phase. Preferably, the dual phase composition is a dual phase chewing gum.

In a further aspect, the invention provides compositions including pharmaceutical compositions comprising any of the ACFP and/or ACP complexes as described above together with a compound that provides monofluorophosphate and a pharmaceutically-acceptable carrier. Such compositions may be selected from the group consisting of dental, anticariogenic compositions and therapeutic compositions. Dental compositions or therapeutic compositions may be in the form of a gel, liquid, solid, powder, cream or lozenge. Therapeutic compositions may also be in the form of tablets or capsules. In one embodiment, the ACP and/or ACFP complexes and compound providing calcium monofluorophosphate are substantially the only remineralizing active components of such a composition. For example, a crème formulation may be employed containing: water; glycerol; CPP-ACP; calcium monofluorophosphate; D-sorbitol; silicon dioxide; sodium carboxymethylcellulose (CMC-Na); propylene glycol; titanium dioxide; xylitol; phosphoric acid; guar gum; zinc oxide; sodium saccharin; ethyl p-hydroxybenzoate; magnesium oxide; butyl p-hydroxybenzoate and propyl p-hydroxybenzoate.

The invention further includes a formulation described above provided together with instructions for its use to treat or prevent any one or more of dental caries or tooth decay, dental erosion, hypersensitivity and fluorosis.

In one embodiment, the active components of the composition consist essentially of the compound that provides calcium monofluorophosphate and stabilized ACP and/or ACFP. It is believed, without being bound by any theory or mode of action, that the stabilized ACP and/or ACFP and the compound that provides calcium monofluorophosphate are central to the therapeutic or preventative effect of the above embodiments of the invention, and thus embodiments consisting essentially of those components (with carriers, excipients and the like as required) are included within the scope of the invention.

The invention also relates to a kit for the treatment or prevention of one or more of dental caries, fluorosis and dental erosion including (a) a compound that provides calcium monofluorophosphate and (b) a CPP-ACP or CPP-ACFP complex in a pharmaceutically acceptable carrier. Desirably, the kit further includes instructions for their use for the mineralization of a dental surface in a patent in need of such treatment. The instructions may describe the use of the kit to treat or prevent one or more of each of dental caries, tooth decay, dental erosion and fluorosis. In one embodiment, the agent and the complex are present in suitable amounts for treatment of a patient.

In a further aspect, there is provided a method of treating or preventing one or more of each of dental caries, tooth decay, dental erosion and fluorosis, comprising the steps of administering a compound that provides calcium monofluorophosphate to the teeth of a subject followed by administering an ACP or ACFP complex or composition. Topical administration of the complex is preferred. The method preferably includes the administration of the complex in a formulation as described above.

In a further aspect there is provided the use of a compound that provides calcium monofluorophosphate and a stabilized amorphous calcium phosphate (ACP) or amorphous calcium fluoride phosphate (ACFP) in a manufacture of a composition for the treatment and/or prevention of one or more of dental caries, tooth decay, dental erosion and fluorosis.

In a further aspect there is provided a composition comprising as an active agent a compound that provides calcium monofluorophosphate and a stabilized amorphous calcium phosphate (ACP) or amorphous calcium fluoride phosphate (ACFP) for mineralizing a dental surface or sub-surface. Typically, mineralizing a dental surface or subsurface is for the treatment and/or prevention of one or more of dental caries, tooth decay, hypersensitivity, dental erosion and fluorosis.

According to a further aspect of the invention there is provided a composition for dental restoration, including a dental restorative material to which has been added a composition of the invention. The base of the dental restorative material can be a glass ionomer cement, a composite material or any other restorative material which is compatible. It is preferred that the amount of stabilized ACP or ACFP, preferably CPP-ACP complex or CPP-ACFP complex, included in the dental restorative material is 0.01-80% by weight, preferably 0.5-10% and more preferably 1-5% by weight. The dental restorative material of this invention which contains the above mentioned agents may be prepared and used in various forms applicable to dental practice. The dental restorative material according to this embodiment may further include other ions, eg. antibacterial ions Zn²⁺, Ag⁺, etc or other additional ingredients depending on the type and form of a particular dental restorative material. It is preferable that the pH of dental restorative material according to this embodiment be between 2-10, more preferably 5-9 and even more preferably 5-7. It is preferable that the pH of the dental restorative material containing the CPP-ACP complex or ACFP complex be in the range of about 2 to 10, more preferably in the range of about 5 to 9 and even more preferably in the range of about 5 to 7.

According to a further aspect of the invention there is provided a varnish including a compound that provides monofluorophosphate and a mineralizing agent. Preferably, the mineralizing agent is any stabilized ACP and/or ACFP complex as described herein and a compound that provides calcium monofluorophosphate.

It will be clearly understood that, although this specification refers specifically to applications in humans, the invention is also useful for veterinary purposes. Thus in all aspects the invention is useful for domestic animals such as cattle, sheep, horses and poultry; for companion animals such as cats and dogs; and for zoo animals.

The invention will now be further described with reference to the following non-limiting examples.

One example of a mineralizing composition or agent comprises the following (in decreasing order of proportion):

water

glycerol

CPP-ACP

D-sorbitol

silicon dioxide

sodium carboxymethylcellulose (CMC-Na)

propylene glycol

titanium dioxide

xylitol

phosphoric acid

guar gum

zinc oxide

sodium saccharin

ethyl p-hydroxybenzoate

magnesium oxide

butyl p-hydroxybenzoate

propyl p-hydroxybenzoate

Such a composition is available from GC corporation under the name Tooth Mousse™ This is suitable for use after a compound that provides calcium monofluorophosphate is added and is in the form of a paste or crème to facilitate its retention on teeth for a suitable period. Alternatively, this mineralizing composition may contain a compound that provides calcium monofluorophosphate.

### Example 1

### Preparation of CPP-ACFP and CPP-ACP Solutions

Stock solutions of 3.25M CaCl₂, 1.25 M Na₂HPO₄, 1M NaOH and 1M NaF were added in approximately thirty aliquots to a 10-15% w/v tryptic digest of casein until a final concentration of approximately 78 mM Ca²⁺, 48 mM phosphate and 12 mM fluoride concentrations were obtained. The solutions were added slowly (that is, less than approximately 1% volume addition per minute). An aliquot of the phosphate solution was added first, followed by an aliquot of the calcium solution. The pH was maintained at between about pH 9.0 and 5.5 using the NaOH with thorough mixing. The sodium hydroxide solution was added automatically by a pH stat with the addition of the hydroxide ions usually being after each addition of the calcium ions. After addition of the calcium ions, phosphate ions, hydroxide ions and fluoride ions the solution was filtered through a 0.1 micron filter to concentrate 1-2 fold. The retentate may be washed with water to remove salts and inactive (and bitter tasting) peptides if desired. CPP-ACP solutions were prepared as above without the addition of fluoride.

### Example 2

### Determining Loosely and Tightly Bound Calcium and Phosphate

At the completion of the titration and filtration for each pH in Example 1, a sample of each retentate was taken and less than 10% collected as a filtrate using a 3000 molecular weight cut-off Centriprep 3 ultrafiltration membrane. The Centripreps containing the samples were centrifuged at 1,000 g for 15 min in a Beckman J2-21 centrifuge using a JA 10.5 rotor. The original sample before Centriprep centrifugation and a sample of the filtrate after Centriprep centrifugation were taken for analysis of calcium, phosphate and fluoride concentrations. The analysis of the original sample gave total calcium, phosphate and fluoride ion concentrations and the analysis of the filtrate gave loosely bound calcium, phosphate and fluoride concentrations. The difference between the total and loosely bound concentrations is the tightly bound concentration of Ca, Pi and F by the CPP.

### Example 3

### Preparation of CPP-ACFP and CPP-ACP Solutions

Recaldent™ (CPP-ACP) was purchased from Recaldent Pty Ltd, Victoria, Australia. The product (#841117) contained 14.3% calcium, 22.3% phosphate and 47% casein phosphopeptide on a weight basis. The product was dissolved at 0.5% and adjusted to pH 5.5 by the addition of HCl. Calcium and phosphate ions were then added by titrating 3.25 M CaCl₂ and 2M NaH₂PO₄ while keeping the pH at 5.5 with the addition of 2.5 M NaOH. The titration of calcium and phosphate ions was continued until the solution became translucent. The concentration of calcium and phosphate added was recorded. The solution may also be formed by titrating calcium and phosphate ions into a 0.5% CPP-ACP solution and letting the pH fall to 5.5 by the addition of further calcium phosphate.

TABLE 2
Calcium and phosphate levels of
normal and superloaded CPP-ACP

| | Calcium | | Phosphate | |
|---|---|---|---|---|
| | | mol/mol | | mol/mol |
| | mmol/L | CPP | mmol/L | CPP |
| Normal 0.5% w/v CPP-ACP | 17.8 | 22.8 | 11.6 | 14.8 |
| Superloaded 0.5 w/v CPP-ACP | 37.8 | 48.3 | 23.6 | 30.2 |
| (sCPP-ACP) | | | | |

These results demonstrate that CPP-ACP can be superloaded with calcium and phosphate ions to produce thermodynamically stable complexes in a metastable solution.

### Example 4

### Preparation of a Formulation of CPP-ACP and Calcium Phosphate

In another example Recaldent™ (CPP-ACP) powder was dry blended with CaHPO₄ powder in the ratio CPP-ACP:CaHPO₄ equals 1:10 on a weight basis. This powder was then added to sugar-free gum and toothpaste formulations at 1-5% w/w.

### Example 5

This example and those following describe exemplary formulations that include a compound that provides calcium monofluorophosphate—this may be calcium monofluorophosphate per se or a compound or composition that produces calcium monofluorphosphate when exposed to an aqueous solution.

A topical crème may be produced in accordance with the present invention having the following ingredients:

Water

glycerol

Stabilized ACP and/or ACFP

A compound that provides calcium monofluorophosphate

D-sorbitol

sodium carboxymethylcellulose (CMC-Na)

propylene glycol

silicon dioxide

titanium dioxide

xylitol

phosphoric acid

sodium fluoride

flavouring

sodium saccharin

ethyl p-hydroxybenzoate

propyl p-hydroxybezoate

butyl p-hydroxybenzoate

### Example 6

A mouthrinse formulation be produced in accordance with the present invention having the following composition:

Water

Alcohol

Poloxamer 407

Sodium Lauryl Sulphate

Stabilized ACP and/or ACFP

A compound that provides calcium monofluorophosphate

Sodium Fluoride

Flavours

Sodium Saccharin

Ethyl p-hydroxybenzoate

Propyl p-hydroxybenzoate

Butyl p-hydroxybenzoate

### Example 7

A sugar-free chewing gum formulation be produced in accordance with the present invention having the following composition:

Crystalline sorbitol/mannitol/xylitol

Gum base

Calcium carbonate

Glycerine

Stabilized ACP and/or ACFP

A compound that provides calcium monofluorophosphate

Sodium Fluoride

Flavour oil

Water

### Example 8

### Preparation of Enamel Subsurface Lesions and Fabrication of Intra-Oral Appliances

Sound relatively planar buccal and lingual surfaces free of cracks, stains and fluorotic lesions (as viewed under a dissecting microscope) were selected from extracted human third molars obtained from the Royal Dental Hospital of Melbourne and treated for at least two weeks in 10% phosphate buffered (pH 7.0) formalin. The teeth were rinsed thrice with Milli-Q water and polished wet to a mirror finish using Soflex (3M) discs on a slow speed contra-angle dental handpiece. Each polished surface was then sawn from the tooth as a slab measuring approximately 8×4 mm, using a water-cooled diamond blade saw (Struers, Denmark). The whole slab was then covered with acid-resistant nail varnish except for two (occlusal and gingival) mesiodistal windows as described by Shen et al. (2001) each measuring 1×7 mm, separated from each other by 1 mm. Subsurface demineralized lesions were created in the enamel windows by immersing each slab in 40 mL of unagitated demineralization buffer containing 80 mL/L Goodrite K-702 polyacrylate solution (Lubrizol Advanced Materials Inc, Cleveland Ohio, USA) (White, 1987), 500 mg/L hydroxyapatite (Bio-Gel HTP, Bio-Rad Laboratories, Richmond, CL), 0.1 mol/L lactic acid (Ajax Chemicals, Auburn, NSW), pH 4.8, for four days at 37° C. After two days the slabs were removed from the buffer, rinsed thrice with Milli-Q water, blotted dry and placed into fresh demineralization buffer for another two days then again rinsed and dried. After demineralization, each enamel slab was sawn through the midline of each window into two 4×4 mm half-slabs and the cut surface of each half-slab covered with nail varnish. One half-slab of each pair was retained as the demineralization control and stored in a labeled 1.7 ml microcentrifuge tube together with a drop of Milli-Q water, to create a humidified environment. The other enamel half-slab of the pair was inset into an intra-oral appliance as described below with the subsurface enamel lesions exposed but recessed 1 mm below the surface of the appliance to create a plaque trap. Care was taken to keep the windows free of wax. Each two half-slab pairs were assigned randomly to either control or treatment. Four enamel half-slabs were inset into each appliance, two on each side in the bilateral troughs (Reynolds et al., 2003).

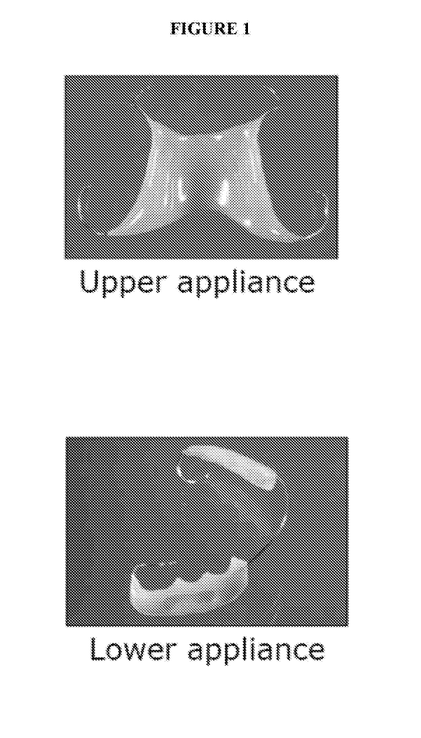

Removable mid-palatal acrylic appliances covering the palate from the first premolar to the last tooth in the arch were produced for each subject as described previously (Iijima et al., 2004). The base of the appliance was retained in the mouth by four narrow-gauge stainless steel circumferential clasps. The appliances were designed with bilateral troughs (15 mm long, 7 mm wide, 3 mm deep) cut into the base and designed to house the enamel slabs. The enamel slabs were retained by sticky wax as described by Iijima et al. (2004) to produce a 1 mm deep trough above the enamel surface to allow plaque to establish and be retained. The appliances used in the clinical trial below are shown in FIG. 1.

### Example 9

### Preparation of Formulations Containing CPP-ACP and Calcium Monofluorophosphate

The preparations and nomenclature used in Tables 3 and 4 and FIG. 2 below are now explained. The preparation used in these studies were prepared based on the formulation for Tooth Mousse™ containing 10% CPP-ACP at pH 7.0. For example, as shown in Table 3, TM 1450 MFP, is a Tooth Mousse formulation containing 10% CPP-ACP at pH 7.0 modified such that it does not contain any other fluoride containing compound except calcium monofluorophosphate at 1450 ppm F. In other words, the MFP refers to calcium monofluorphosphate at an amount of 1450 ppm F. Calcium monofluorophosphate was formed by adding 76.3 μmol of disodium monofluorophosphate per g together with 80.0 μmol of calcium chloride (CaCl₂) per g at pH 7.0 and mixed thoroughly to produce calcium monofluorophosphate. Other sources of monofluorophosphate, for example as described herein, and other sources of calcium, for example as described herein, could be used to produce calcium monofluorophosphate. Also, calcium monofluorophosphate could be formed at other pH, for example 4.0 to 9.0, preferably, 5.0 to 7.0, even more preferably about 7.0. In addition, reference to, for example, MFP 1450 in Table 3 a Tooth Mousse formulation modified such that it does not contain CPP-ACP and does not contain any other fluoride containing compound except calcium monofluorophosphate.

### Example 10

### The Clinical Study was a Double-Blind, Randomized, Controlled, Cross-Over In Situ Trial to Assess the Ability of the Experimental Formulations to Remineralize Enamel Subsurface Lesions

Approval for the study was obtained from The University of Melbourne Human Research Ethics Committee. The study was conducted in Melbourne, Australia. Twelve healthy participants (6 males and 6 females) with ages ranging from 30-50 were recruited for the study and provided informed consent. The minimum number of participants required for the study was based on our previous studies using the same in situ model and cross-over design (Cai et al., 2009; Walker et al., 2010). All participants had at least 22 natural teeth with no active caries, periodontal disease or other oral pathology and none were using antibiotics or medications known to affect salivary flow rate. Saliva (unstimulated) was collected from each participant by tilting the head and allowing saliva to flow into a 15 ml pre-weighed tube for a period of two minutes. Saliva was stimulated by chewing sugar-free gum for two minutes and collected during this period as for unstimulated saliva. The saliva volume was determined by weight measurement.

The products were randomized into coded treatments. One investigator generated the random allocation sequence, enrolled the participants and assigned participants to coded products. The randomization code was held independently of the participants and study investigators. A product to water slurry (1 g product plus 4 ml of distilled, deionised water vortexed for 5 min) was prepared fresh in a coded plastic tube prior to treatment each day. Each participant with the appliance inserted rinsed 5 ml of slurry for 60 seconds. This was performed 4 times a day for 10 days at the following times: 10:00 am, 11:30 am, 2:00 pm, and 3:30 pm. The participants were instructed to remove the appliances before eating and drinking and performing oral hygiene procedures. When the appliances were removed they were stored in a sealed moist plastic bag at room temperature. Participants were instructed not to remove their appliances for at least one hour after each treatment. When wearing the appliance participants were instructed not to eat or drink anything except water. Appliances were cleaned with distilled water only and not over the inset enamel slabs. The participants crossed over to each of the treatments after at least a one week washout period. Participants kept a diary of product use. No alterations were made to the participants' diet and oral hygiene procedures for the duration of the study. The participants were supplied with a 1000 ppm F (NaF) toothpaste (Colgate) and toothbrush (Colgate) for their normal oral hygiene procedures but used no other fluoride products during the study except the study products. After completion of each treatment period the enamel slabs were removed from the appliances, rinsed with Milli-Q water and stored in a humidified environment prior to analysis.

### Example 11

Sectioning and Microradiography

The remineralized enamel half-slab was placed together with its demineralized control half-slab into freshly poured transparent cold curing methacrylate resin (Paladur, Heraeus Kulzer, Germany) with the lesion windows parallel (Iijima et al., 2004). The resin vial was marked at the top corner to identify the test and control blocks and the resin was allowed to set at room temperature overnight. Sections approximately 200 μm thick were cut from the embedded blocks perpendicular to the lesion surface through the midline of the lesions using an internal annulus saw microtome (Leica 1600, Leica, Germany). The sections were then lapped down to 80±5 μm using a RotoPol-21/RotoForce4 lapping instrument (Struers, Denmark) with 1200 and 2400 grit lapping paper. Each section, which contained the remineralized lesion and the demineralized control lesion from the same enamel slab, was radiographed along with an aluminium stepwedge of 10×14 μm thick increments using Microchrome High Resolution glass plates (Type 1A, Microchrome, USA) and nickel filtered copper K radiation at 20 kV, 10 mA for 6 minutes. The radiographic apparatus used has been described previously by Malcolm (1972). Each glass plate was developed in Microchrome Developer D5 (1:4 dilution, Microchrome, USA) for 4 minutes, placed into glacial acetic acid stop bath for 30 seconds and then fixed in Microchrome Fixer F4 (1:4 dilution, Microchrome, USA) for 4 minutes.

Microdensitometry

Radiographic images of the lesions were viewed via transmitted light through a Dialux 20 microscope (Ernst Leitz Welzlar, Germany). The images were then acquired by a digital camera (Spot Insight, Diagnostic Instruments, Inc., MI, USA) analyzed by using imaging software Optimate version 5.2. Images of the lesions and the neighboring areas of sound enamel were scanned using the programs line luminance function that gives readings in grey values between 0 and 256. An area free of artifacts or cracks was selected for analysis. Each scan comprised 200 readings taken from the tooth surface through the lesion to sound enamel. The aluminium stepwedge image on each slide was scanned and the averaged step grey value readings were plotted against aluminium thickness. The readings of the tooth section image lay within the linear portion of the stepwedge curve and linear regression was used to convert the grey value data into values of equivalent thickness of aluminium. The section thickness was measured and the vol % mineral data computed using the equation of Angmar (1963) and the linear absorption coefficients of aluminium, organic matter plus water and apatitic mineral (131.5, 11.3 and 260.5 respectively). The image of the median sound enamel between the two lesions (gingival and occlusal) was scanned six times and averaged to give a control sound-enamel densitometric profile. The lesion images (remineralization windows and demineralization control window) to the gingival and occlusal side of the median sound enamel were similarly scanned six times, as close as possible to the median strip but avoiding any irregularities commonly found at the lesion edges. The values for the multiple scans were averaged, and the volume % mineral profiles were computed. These measurements were repeated for the four enamel blocks used by each participant for each treatment and then averaged to provide a single value for that participant and treatment.

### Example 12

Remineralization Data Analysis

The % mineral profile of each enamel block's demineralized and remineralized lesion was compared with the median sound enamel % mineral profile of the same section. The difference between the areas under the densitometric profile of the demineralized lesion and the median sound enamel, calculated by trapezoidal integration, is represented by ΔZd. The difference between the areas under the densitometric profile of the remineralized lesion and the median sound enamel, calculated by trapezoidal integration, is represented by ΔZr. These parameters were then converted to % change values after remineralization, as such, % remineralization (% R) represents the % change in ΔZ values: % R=(ΔZd−ΔZr)/ΔZd×100.

The primary outcome measure was percentage mineral gain (% R). This was compared across treatments using a repeated-measures analysis of variance (ANOVA) model with factors for treatment sequence, treatment period and treatment type (Jones and Kenward, 2003). The secondary outcome measure was post-product rinse/saliva calcium, inorganic phosphate and fluoride concentrations and were compared across treatment products using repeated-measures ANOVA models with factors for treatment sequence, treatment period and treatment type. Post hoc comparisons of treatment differences were performed on the marginal means using the Sidak adjustment for multiple comparisons (SPSS, 2008). ANOVA assumptions were checked using residual and normal probability plots. The unit of analysis was the participant. The outcome measures were determined and the resultant values averaged by participant and treatment period. p values less than 0.05 were regarded as being statistically significant. All analyses were conducted using either SPSS (version 17, SPSS Inc, Chicago, Ill., USA) or Stata (Version 10, Stata Corp LP, College Station, Tex., USA) statistical software.

Post-rinse samples from the clinical trial described in Example 10 were centrifuged at 1,000 g for 15 min. Samples of the supernatants were diluted with distilled/deionised water and acidified with 0.01N HNO3 thoroughly mixed and transferred into a 10 ml syringe fitted with a 0.2 μm filter (Minisart, Sartorious, Victoria, Australia). Results are shown in Table 5.

### Example 13

Product Analyses

Total (acid soluble) calcium, phosphate and fluoride levels of products were determined by adding 1 g of product to 19 ml of 1N HNO3 in a 50 ml centrifuge tube and mixed overnight. This slurry was then diluted further with 0.01N HNO3 as appropriate and analysed for ions using the ion chromatography system after centrifugation and filtration as described below. Water soluble calcium, phosphate and fluoride levels were determined by adding 1 g of product to 19 ml of deionized water in a 50 ml centrifuge tube and mixed overnight. The sample was then centrifuged for 1,000 g for 15 min at room temperature. A sample of the supernatant was added to an equal volume of 0.01N HNO3 and filtered through a 0.2 μm filter.

Filtrates were analysed for inorganic ions (calcium, inorganic phosphate and fluoride) using atomic absorption spectroscopy or an automatic ion chromatography system (Dionex Corporation, CA, USA) equipped with two columns for both cation (IonPac CS12) and anion (IonPac AS18) analysis using two conductivity detectors (ICS 3000).

A combined seven anion standard and a combined six cation standard were used to calibrate the instrument.

TABLE 3
Soluble and total calcium, inorganic phosphate,
fluoride contents of experimental products

| | μmol/g paste | | |
|---|---|---|---|
| Product | Ca | Pi | Fluoride |
| F 1450 (soluble) | nd | nd | 69.37 ± 1.32 |
| F 1450 (total) | nd | nd | 73.07 ± 1.36 |
| MFP 1450 (soluble) | 80* | nd | 70.08 ± 4.94 |
| MFP 1450 (total) | 80* | nd | 68.95 ± 0.80 |
| TM 1450 NaF | 405.63 ± 16.76 | 180.61 ± 4.86 | 67.47 ± 1.08 |
| (soluble) | | | |
| TM 1450 NaF (total) | 413.48 ± 14.30 | 210.18 ± 8.49 | 72.16 ± 3.04 |
| TM 1450 MFP | 418.55 ± 20.32 | 180.81 ± 12.08 | 68.24 ± 3.87 |
| (soluble) | | | |
| TM 1450 MFP (total) | 483.49 ± 28.20 | 244.35 ± 2.04 | 67.60 ± 1.55 |

nd, not detected under the condition tested.
*approximately.

### Example 14

Clinical Trial Result

The results of the clinical trial are presented in Table 4, Table 5 and FIG. 2. These results show the products (i.e. Tooth Mousse ((TM)) containing calcium monofluorophosphate (CaMFP) were significantly better (Repeated Measures Analysis of Variance significance: p<0.05) than those containing sodium fluoride at the same fluoride concentration (1450 ppm F) in remineralizing enamel subsurface lesions in situ.

Mean and standard error§for LDd, LDr, LDd-LDr, ΔZd, ΔZd−ΔZr and percent remineralisation (% R) measured separately for upper and lower appliances for each treatment and for all treatments combined. Differences between parameters for the two appliances (FIG. 1) were analysed using paired t-tests except those marked * where Wilcoxon Signed Ranks Sum tests with a Bonferroni correction were used. For each parameter, there was no difference between upper and lower appliances (p>0.05).

TABLE 4
Remineralisation of enamel subsurface lesions in situ by NaF or CaMFP with and without CPP-ACP

| Treatment | Appliance | LDd | LDr | LDd − LDr | ΔZd | ΔZd − ΔZr | % R |
|---|---|---|---|---|---|---|---|
| 1450 ppm F | Upper | 103.55 ± 3.86 | 101.59 ± 3.71 | 1.97 ± 1.23 | 2728.90 ± 204.64 | 290.73 ± 17.17 | 10.76 ± 0.26* |
| as NaF | Lower | 109.10 ± 2.97 | 103.46 ± 2.41 | 5.64 ± 1.47 | 3002.24 ± 130.46 | 306.33 ± 16.32 | 10.19 ± 0.22* |
| 1450 ppm F | Upper | 121.26 ± 1.90 | 102.21 ± 1.94* | 19.05 ± 1.29 | 3587.25 ± 156.96 | 1146.46 ± 55.51 | 31.92 ± 0.38 |
| as NaF plus | Lower | 120.16 ± 3.14 | 100.35 ± 1.85* | 19.81 ± 1.47 | 3443.09 ± 138.22 | 1104.28 ± 48.21 | 32.04 ± 0.19 |
| CPP-ACP | | | | | | | |
| (TM NaF) | | | | | | | |
| 1450 ppm F | Upper | 115.05 ± 2.60 | 111.44 ± 2.39 | 3.61 ± 1.13 | 3035.93 ± 151.79 | 377.06 ± 19.54 | 12.44 ± 0.30 |
| as CaMFP | Lower | 114.57 ± 1.99 | 108.20 ± 1.78 | 6.37 ± 0.85 | 3297.01 ± 99.45 | 420.86 ± 15.28 | 12.77 ± 0.30 |
| 1450 ppm F | Upper | 116.64 ± 2.28 | 98.02 ± 2.21 | 18.62 ± 1.98 | 3102.27 ± 100.68 | 1078.14 ± 27.47 | 34.83 ± 0.63 |
| as CaMFP | Lower | 120.08 ± 3.37 | 100.73 ± 2.46 | 19.35 ± 2.03 | 3267.39 ± 163.61 | 1136.16 ± 59.99 | 34.75 ± 0.23 |
| plus CPP- | | | | | | | |
| ACP (TM | | | | | | | |
| CaMFP) | | | | | | | |

TABLE 5
Saliva post rinse calcium, inorganic phosphate, fluoride and monofluorophosphate
contents for paste products tested in situ.

| | Ca | Pi | F⁻ | MFP | Total (F + MFP) |
|---|---|---|---|---|---|
| Product | (μmol/ml) | (μmol/ml) | (ppm) | (ppm) | (ppm) |
| F 1450 (NaF) | 2.90 ± 1.52 | 1.01 ± 0.48 | 151.11 ± 12.79 | nd | 151.11 ± 12.79 |
| MFP 1450 | 0.73 ± 0.45 | 1.32 ± 0.53 | 11.87 ± 3.32 | 180.22 ± 20.07 | 192.08 ± 20.08 |
| (CaMFP) | | | | | |

nd, not detected under the condition tested.

The results in Table 5 show that the monofluorophosphate present in the products as calcium monofluorophosphate remained as monofluorophosphate in the oral cavity, whereas the sodium fluoride completely dissociated into free fluoride ions in the oral cavity of the subjects. Specifically, in the post rinse saliva of individuals that used the Tooth Mousse product containing calcium monofluorophosphate, almost all the monofluorophosphate remained as monofluorophosphate without dissolving into free fluoride ions (i.e. of the 192.08±20.08 ppm fluoride detected in the product, 180.22±20.07 ppm monofluorophosphate was detected in the saliva post rinse).

### REFERENCES

Reynolds E C (1998). Anticariogenic complexes of amorphous calcium phosphate stabilized by casein phosphopeptides: a review. Spec Care Dentist 18:8-16.
Shen P, Cai F, Nowicki A, Vincent J, Reynolds E C (2001). Remineralization of enamel subsurface lesions by sugar-free chewing gum containing casein phosphopeptide-amorphous calcium phosphate. J Dent Res 80:2066-70.
Angmar B, Carlstrom D, Glas J E (1963). Studies on the ultrastructure of dental enamel. IV. The mineralization of normal human enamel. J Ultrastruct Res 8:12-23.
Cai F, Shen P, Walker G D, Reynolds C, Yuan Y, Reynolds E C (2009). Remineralization of enamel subsurface lesions by chewing gum with added calcium. J Dent 37:763-768.
Iijima Y, Cai F, Shen P, Walker G, Reynolds C, Reynolds E C (2004). Acid resistance of enamel subsurface lesions remineralized by a sugar-free chewing gum containing casein phosphopeptide-amorphous calcium phosphate. Caries Res 38:551-556.
Jones B, Kenward M G (2003). Design and analysis of cross-over trials. 2nd ed. Boca Raton, Fla., USA: Chapman and Hall/CRC.
Malcolm A S (1972). A microradiographic apparatus. Aust Dent J 17:454-460.
Reynolds E C, Cai F, Shen P, Walker G D (2003). Retention in plaque and remineralization of enamel lesions by various forms of calcium in a mouthrinse or sugar-free chewing gum. J Dent Res 82:206-211.
SPSS (2008). Statistics Base 17.0 User's Guide Chicago, Ill., USA: SPSS Inc.
Walker G D, Cai F, Shen P, Adams G G, Reynolds C, Reynolds E C (2010). Casein phosphopeptide-amorphous calcium phosphate incorporated into sugar confections inhibits the progression of enamel subsurface lesions in situ. Caries Res 44:33-40.
White D J (1987). Use of synthetic polymer gels for artificial carious lesion preparation. Caries Res 21:228-242.

1. A composition for mineralizing a dental surface or sub-surface comprising calcium monofluorophosphate and a mineralizing agent.

2. A composition according to claim 1, wherein the calcium monofluorophosphate is in an amount of about 200 ppm to about 5000 ppm F.

3. A composition according to claim 2, wherein the calcium monofluorophosphate is in an amount of about 400 ppm to about 1500 ppm F.

4. A composition according to claim 3, wherein the calcium monofluorophosphate is in an amount of about 1000 ppm to about 1450 ppm F.

5. A composition according to any one of claims 1 to 4, wherein the mineralizing agent is stabilized amorphous calcium phosphate (ACP) and/or amorphous calcium fluoride phosphate (ACFP).

6. A composition according to any one of claims 1 to 5, wherein the composition is selected from the group consisting of a toothpaste, toothpowder, liquid dentifrice, mouthwash, mouthrinse, mouth spray, varnish, dental cement, troche, chewing gum, lozenge, dental paste, gingival massage cream, gargle tablet, dairy product and other foodstuffs.

7. A composition according to claim 6, wherein the composition is a toothpaste.

8. A composition according to claim 6, wherein the composition is a varnish.

9. A composition according to claim 6, wherein the composition is a dental cement.

10. A composition according to claim 6, wherein the composition is a mouthwash.

11. A composition according to any one of claims 1 to 10, for use in a domestic animal, companion animal or zoo animal.

12. A method of mineralizing a dental surface or sub-surface comprising contacting the dental surface or subsurface with calcium monofluorophosphate and a mineralizing agent.

13. A method according to claim 12, wherein the calcium monofluorophosphate is in an amount of about 200 ppm to about 5000 ppm F.

14. A method according to claim 12, wherein the calcium monofluorophosphate is in an amount of about 400 ppm to about 1500 ppm F.

15. A method according to claim 12, wherein the calcium monofluorophosphate is in an amount of about 1000 ppm to about 1450 ppm F.

16. A method according to any one of claims 12 to 15, wherein the calcium monofluorophosphate is provided in a composition and the calcium monofluorophosphate is present in the composition prior to administration to an oral cavity.

17. A method according to any one of claims 12 to 16, wherein the dental surface is dental enamel.

18. A method according to any one of claims 12 to 16, wherein the dental surface is a lesion in the enamel, caused by caries, dental erosion or fluorosis.

19. A method according to any one of claims 12 to 18, wherein the mineralizing agent is stabilized amorphous calcium phosphate (ACP) and/or amorphous calcium fluoride phosphate (ACFP).

20. A method according to claim 19, wherein the mineralizing agent is casein phosphopeptide stabilized amorphous calcium phosphate (ACP) and/or amorphous calcium fluoride phosphate (ACFP).

21. A method according to any one of claims 12 to 20, wherein the dental surface of subsurface is of a domestic animal, companion animal or zoo animal.
